(19) United States
(12) Patent Application Publication
(10) Pub. No.: US 2020/0138822 A1
(43) Pub. Date: May 7, 2020

(54) TOPICAL COMPOSITIONS COMPRISING PENTOXIFYLLINE AND METHODS OF TREATMENT USING THE SAME

(71) Applicant: Tim COTE, Washington, DC (US)

(72) Inventor: Tim COTE, Washington, DC (US)

(21) Appl. No.: 16/676,167

(22) Filed: Nov. 6, 2019

Related U.S. Application Data
(60) Provisional application No. 62/756,401, filed on Nov. 6, 2018.

(51) Int. Cl. A61K 31/522; A61K 9/00; A61K 47/14; A61K 47/38; A61K 47/10; A61P 1/04

(52) U.S. Cl. CPC A61K 31/522; A61K 9/0034; A61K 9/006; A61P 1/04; A61K 47/38; A61K 47/10; A61K 47/14

(57) ABSTRACT

The disclosure provides topical compositions comprising pentoxifylline and methods of using the same.

### CROSS-REFERENCE TO RELATED APPLICATION

This application claims priority to U.S. Provisional Patent Application No. 62/756,401 filed on Nov. 6, 2018, which is hereby incorporated by reference in its entirety.

### BACKGROUND

Behçet's Disease (BD) is a complex, chronic, relapsing, multi-system inflammatory disorder that is characterized by oral ulcers, genital ulcers, ocular lesions, and skin lesions. BD is usually a diagnosis of exclusion for which international diagnostic criteria have been developed. A genetic/immunologic basis for the disorder is suggested by its association with human leukocyte antigen HLA-B51, which occurs in around 60% of BD patients. There is a concentrated prevalence of BD along the Silk Road, an ancient trading route extending from Japan to Turkey, with Turkey having the highest prevalence of BD. Vasculitis is the dominant pathologic lesion, accordingly there are reports of clinical responsiveness to immune modulatory agents.

The most common symptom of BD is recurrent oral ulcers, which profoundly affects patients' quality of life. The oral ulceration in BD is indistinguishable from recurrent aphthous stomatitis. They arise in crops as discrete, multiple aphthous ulcers; they are usually painful, persist for an average of two weeks, and subside without scaring. OTEZLA® (apremilast) is the only therapy approved by the U.S. Food and Drug Administration (FDA) for oral ulcers associated with BD, and there is no cure or therapy approved by the FDA for genital ulcers associated with BD. Otherwise, the treatment of recurrent oral or genital ulcers that are associated with BD is palliative.

Various preparations may be used to treat oral or genital ulcerations. For example, carbenoxolone sodium can be helpful in promoting healing of mild oral lesions, lozenges and oral pastes containing corticosteroids are used for treating apthous non-specific ulcers, salicylates can be used in mild inflammatory and painful oral lesions, and benzydamine hydrochloride has a topical anti-inflammatory and analgesic effect and is used as a mouthwash or spray for oral ulcerations. Oral ulcerations may be difficult to treat with preparations such as sprays, lozenges, and mouthwash, because the active agent of the preparation is only transiently in contact with the ulcer. Even with gelatin-based pastes, which have a protective effect for non-infected ulcers, it is difficult to keep them on the lesion for a prolonged period. An example of a mucoprotectant is Orabase™, which contains carmellose sodium, pectin, and gelatin. This composition, however, has an uncomfortable gritty mouthfeel.

The American Behçet's Disease Association (ABDA) notes topical triamcinalone acetonide is used to treat oral ulcers and betamethasone ointment is used to treat genital ulcers to relieve pain and discomfort. ABDA notes systemic TNF inhibitors like ENBREL® (etanercept), REMICADE® (infliximab), HUMIRA® (adalimumab), CIMZIA® (certolizumab), or SIMPONI® (golimumab) are used to treat inflammatory conditions associated with BD. In addition, ABDA notes systemic tetracycline (oral only), cochicine, thalidomide, dapsone, or pentoxifylline are used to treat oral or genital ulcers. Also, ABDA notes “Magic Mouthwash” (a compounded mixture of usually at least three of the following ingredients: (1) an antibiotic to kill bacteria around the sore, (2) an antihistamine or local anesthetic to reduce pain and discomfort, (3) an antifungal to reduce fungal growth, (4) a corticosteroid to treat inflammation, or (5) an antacid that helps ensure the other ingredients adequately coat the inside of your mouth) is used to alleviate pain in the oral mucosa. ABDA does not endorse or recommend any of these therapies, listing them as examples only.

Yet none of these treatments listed by the ABDA directly effectively treat the oral or genital sores and are associated with undesirable side effects.

Pentoxifylline is a tri-substituted xanthine derivative that improves the flow properties of blood by decreasing viscosity and has been associated with a variety of associated anti-inflammatory effects, as well as unwanted gastrointestinal and central nervous impairment. Systemic oral preparations of pentoxifylline, however, are only currently approved and marketed for the treatment of patients with intermittent claudication on the basis of chronic occlusive arterial disease of the limbs, under a new drug application (NDA) for TRENTAL®. And systemic pentoxifylline is associated with a high rate of gastrointestinal and central nervous impairment, which may reduce the ability to use the systemic formulation over the required duration of treatment.

### SUMMARY OF VARIOUS ASPECTS OF THE DISCLOSURE

The following presents a summary of one or more aspects of the disclosure in order to provide a basic understanding of such aspects. This summary is not an extensive overview of all contemplated aspects and is intended to neither identify key or critical elements of all aspects nor delineate the scope of any or all aspects. Its purpose is to present some concepts of one or more aspects in a simplified form as a prelude to the more detailed description that is presented later.

The disclosure provides a method of treating Behçet's Disease (BD) comprising topically administering a pharmaceutical composition comprising a therapeutically effective amount of pentoxifylline (PTX), to a human in need of treatment. In some aspects, the pharmaceutical composition is a topical oral or genital mucosal formulation.

The disclosure also provides a topical oral or genital mucosal pharmaceutical composition comprising pentoxifylline, wherein the composition is suitable for direct application of pentoxifylline to BD lesions. In some aspects, the composition comprises: about 5% (w/w) PTX, about 1.3% (w/w) polyethylene oxide, about 3% (w/w) hypromellose, about 0.8% (7:3 w/w) methylparaben:propylparaben, and distilled water q.s. 100% (w/w).

The disclosure provides a method of treating Behçet's Disease (BD) comprising topically administering a pharmaceutical composition comprising a therapeutically effective amount of pentoxifylline (PTX) in conjunction with other oral medications, such as oral colchicine (CLC), to a human in need of treatment. In some aspects, the pharmaceutical composition is a topical oral or genital mucosal formulation.

The disclosure also provides for a method of treating recurrent aphthous stomatitis, comprising topically administering a pharmaceutical composition comprising a therapeutically effective amount of PTX, to a human in need of treatment. In some aspects, the method comprises the administration of topical PTX with other oral medications, such as CLC.

These and other aspects will become more fully understood upon a review of the detailed description, which follows.

### BRIEF DESCRIPTION OF THE DRAWINGS

FIG. 1 shows the mean ulcer index ulcer area by treatment group (PTX+CLC) and (CLC) for the study described in Example 3. At day zero, index ulcer was similar for both groups, but mean ulcer index immediately decreased for PTX+CLC, while first growing then slowly decreasing for CLC-only.

FIG. 2 shows the mean ulcer index ulcer area by treatment group as in FIG. 1, but removes one outlier patient who experienced dramatic ulcer growth with the CLC only treatment.

FIG. 3 shows normalized pain scores for the treatment groups described in Example 3. The normalized pain scores decreased faster and remained lower for PTX+CLC compared to CLC alone.

### DETAILED DESCRIPTION

The disclosure provides topical compositions comprising pentoxifylline (PTX) alone or in combination with colchicine (CLC), and methods of treatment using the same, such as a method of treating oral or genital sores or ulcers caused by Behçet's Disease (BD). The detailed description includes specific details for the purpose of providing a thorough understanding of various concepts. However, it will be apparent to those skilled in the art that these concepts may be practiced without these specific details.

As used herein, the term “about” is defined to be within 10%, or within 5%, or within 0.5%.

As used herein, the terms “topical oral” “topical oral mucosal” are used interchangeably, as are “topical genital” and “topical genital mucosal”. Oral or genital ulceration (mouth or genital ulcers) can be very painful, and the resulting lesions can be mild or severe.

Surprisingly, it is found that application of topical oral or genital composition comprising PTX may accelerate healing of recurrent oral ulcers symptomatic of BD in a clinically-meaningful way. Furthermore, a topical composition comprising PTX (e.g., oral mucosal composition) can become a valuable adjunct to any other systemic therapy for BD, such as CLC. Without wishing to be bound to any particular theory, a topical oral or genital formulation of PTX may maximize local concentrations of PTX delivered to affected mucosal sites while minimizing adverse effects that may otherwise occur from systemic exposure, as well as reducing required dosing.

The disclosure provides a method of treating BD comprising topically administering to a patient in need of treatment a pharmaceutical composition comprising a therapeutically effective amount of PTX. In other aspects, the disclosure provides a method of treating BD comprising topically administering to a patient in need of treatment a pharmaceutical composition comprising a therapeutically effective amount of PTX in conjunction with other oral medications, such as oral colchicine (CLC). The data provided in Example 3 demonstrates, for example, that topical PTX appears to have stronger immediate effects on ulcer healing, faster effects on ulcer clearance, and lessening effects on ulcer number than oral CLC by itself.

PTX is a tri-substituted xanthine derivative designated chemically as 1-(5-oxohexyl)-3,7-dimethylxanthine and is a hemorrheologic agent (an agent that affects blood viscosity possibly by improving the flexibility of red blood cells). PTX is also believed to inhibit tumor necrosis factor alpha (TNF-α). PTX is soluble in water and ethanol, and sparingly soluble in toluene. The CAS Registry Number is 6493-05-6. The chemical structure is below:

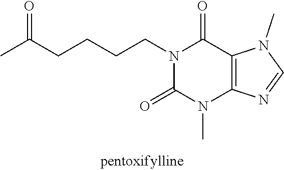 [SMILES: CC(=O)CCCCn1c(=O)c2c(ncn2C)n(C)c1=O]

PTX's major metabolites are Metabolite 1 (1-[5-hydroxyhexyl]-3,7-dimethylxanthine) and Metabolite V (1-[3-carboxypropyl]-3,7-dimethylxanthine), and plasma levels of these metabolites are five and eight times greater, respectively, than PTX.

In some aspects, treatment of BD includes treatment of one or more symptoms of BD, including but not limited to recurrent oral or genital ulcers.

The pharmaceutical compositions described herein are administered topically. In some aspects, the topical formulation is muco-adherent. In some aspects, the composition is administered directly to recurrent oral or genital ulcers. In some aspects, the composition is administered directly to recurrent oral or genital ulcers and immediately peripheral to the recurrent oral or genital ulcers, such as in a partial or whole ring around the oral or genital ulcers. Such rings may partially or wholly concentrically surround the respective oral or genital ulcer. The rings may be directly around the periphery of the ulcer, such as at a distance of no more than about 2 mm radially outward from the periphery of the ulcer.

Without wishing to be bound to any particular theory, it is believed that topical oral or genital administration may maximize local concentrations of PTX delivered to affected mucosal sites while minimizing any adverse effects of systemic administration of PTX. Systemic therapy for BD may include, but is not limited to, treatment for one or more other symptoms of BD.

The disclosure provides for topical compositions comprising PTX. In some aspects, the composition comprises PTX, polyethylene oxide, hypromellose, methylparaben:propylparaben, and distilled water. In particular aspects, the composition comprises about 5% (w/w) PTX, about 1.3% (w/w) polyethylene oxide, about 3% (w/w) hypromellose, about 0.8% (7:3, w/w) methylparaben:propylparaben, and distilled water q.s. 100% (w/w).

In some aspects, the topical pharmaceutical composition is administered in one or more doses of no more than about 5 g each of the pharmaceutical composition, up to 20 g per day (e.g., 10 applications of 2 g each. The topical pharmaceutical composition may be administered in one or more doses of about 0.5 g, about 1 g, about 1.5 g, about 2 g, about 2.5 g, about 3 g, about 3.5 g, about 4 g, about 4.5 g, about 5 g, or any amount in between. In some aspects, the topical pharmaceutical composition is administered in one or more doses of no more than about 5 g each of the pharmaceutical composition. In some aspects, the maximum daily dosage of the topical pharmaceutical composition is no more than about 20 g of the pharmaceutical composition.

In some aspects, the maximum daily dosage of the topical pharmaceutical composition is no more than about 20 g of the topical pharmaceutical composition.

In some aspects, the topical pharmaceutical composition is administered at least twice daily. In some aspects, the topical pharmaceutical composition is administered four times daily.

In some aspects, the PTX treats the symptom of oral ulcers in the patient.

In some aspects, the topical pharmaceutical composition is administered one or more times or at least twice daily, up to 20 g in composition of the total daily dose. In some aspects, the topical pharmaceutical composition is administered four times daily. Four times daily administration of about 5 g of the above composition yields a maximum daily dose of about 1000 mg/day of the active pharmaceutical ingredient (API) PTX. This is less than the total recommended dose of 1200 mg/d for TRENTAL®. As will be known to persons of ordinary skill in the art, patients with fewer lesions may not require the full 5 g dose at each application. It is within the knowledge of those of ordinary skill in the art to adjust or optimize the dose in order to maximize therapeutic effectiveness while minimizing adverse effects, including, but not limited to, those arising from accidental swallowing of topical PTX.

In other embodiments, the disclosure provides a topical oral or genital mucosal pharmaceutical composition comprising: about 5% (w/w) PTX, about 1.3% (w/w) polyethylene oxide, about 3% (w/w) hypromellose, about 0.8% (7:3 w/w) methylparaben:propylparaben, and distilled water q.s. 100% (w/w).

All aspects described with reference to one aspect apply to all other aspects with equal force, and vice versa. The topical pharmaceutical composition is preferably formulated for topical oral or genital administration.

The topical oral or genital formulation contains two inactive ingredients with muco-adhesive properties: hypromellose and polyethylene oxide (The Dow Chemical Company, 2002 and 2003). The first inactive ingredient, hypromellose meets the specifications USP Pharmacopoeia (United States Pharmacopoeia-National Formulary (USP-NF) 2105 and 2107). Furthermore, a similar product that can be used as a possible hypromellose substitute, methyl cellulose, is generally recognized as safe (GRAS) by US FDA (USP-NF 2664, 2665, and 2666). The second inactive ingredient, polyethylene oxide meets the specifications of USP Pharmacopoeia (USP-NF 5503).

In a particular aspect, the topical pharmaceutical composition comprises about 5% (w/w) PTX, about 1.3% (w/w) polyethylene oxide, about 3% (w/w) hypromellose, about 0.8% (7:3 w/w) methylparaben:propylparaben, and distilled water q.s. 100% (w/w) and contains a final drug product concentration of about 50 mg/ml. Application of 5 g of the topical pharmaceutical composition results in the delivery of about 250 mg of PTX in soluble form.

The topical pharmaceutical compositions described herein may be prepared according to any suitable method known in the art. In one non-limiting example, the composition is prepared similarly to another topical cream with 5% PTX (Allen, 2004) but incorporates muco-adherent ingredients, hypromellose and polyethylene oxide, similar to other muco-adherent formulations for intraoral administration (Baloglu et al., 2010, Karavana et al., 2011). PTX is dissolved in appropriate amount of water, and this solution is added in small portions to the wetted gel base containing hypromellose and polyethylene oxide prepared according to the manufacturer's instructions (The Dow Chemical Company, 2002 and 2003). Final consistency is achieved by mixing thoroughly with the aid of a vortex. The composition may be prepared with or without sterile techniques.

The composition may be in any form suitable for topical oral or genital administration, depending on the excipients used, their concentrations, and the conditions of preparation of the composition. Such topical forms include, but are not limited to, creams, gels, ointments, lotions, and pastes. In some aspects, the composition is a topical cream. It is within the knowledge of those of ordinary skill in the art to adjust ingredients, concentrations, and conditions to modify the composition. In other aspects, the composition is formulated for oral, parenteral, sublingual, or buccal administration.

The compositions described herein can additionally include preserving agents such as methyl-, ethyl-, and propyl-hydroxy-benzoates, butylated hydroxytoluene, and butylated hydroxyanisole; sweetening agents; flavoring agents; and coloring agents. Sweetening agents can be used to improve the palatability of the composition by masking any unpleasant tastes it may have. Examples of suitable sweetening agents include, without limitation, compounds selected from the saccharide family such as the mono-, di-, tri-, poly-, and oligosaccharides; sugars such as sucrose, glucose (corn syrup), dextrose, invert sugar, fructose, maltodextrin, and polydextrose; saccharin and salts thereof such as sodium and calcium salts; cyclamic acid and salts thereof dipeptide sweeteners; chlorinated sugar derivatives such as sucralose and dihydrochalcone; sugar alcohols such as sorbitol, sorbitol syrup, mannitol, xylitol, hexa-resorcinol, and the like, and combinations thereof. Hydrogenated starch hydrolysate, and the potassium, calcium, and sodium salts of 3,6-dihydro-6-methyl-1-1,2,3-oxathiazin-4-one-2,2-dioxide may also be used. Of the foregoing, sorbitol, mannitol, and xylitol, either alone or in combination, are preferred sweetening agents. The compositions of the present invention can comprise from about 0% to about 80% by weight of the sweetening agent.

Flavoring agents can also be used to improve the palatability of the compositions described herein. Examples of suitable flavoring agents include, without limitation, natural and/or synthetic (i.e., artificial) compounds such as peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, chocolate (e.g., white, milk, dark), vanilla, caramel, coffee, hazelnut, combinations thereof, and the like. Coloring agents can be used to color code the composition, for example, to indicate the type and dosage of the therapeutic agent therein. Suitable coloring agents include, without limitation, natural and/or artificial compounds such as FD & C coloring agents, natural juice concentrates, pigments such as titanium oxide, silicon dioxide, and zinc oxide, combinations thereof, and the like. The compositions of the present invention can comprise from about 0% to about 10% by weight of the flavoring and/or coloring agent, preferably from about 0.1% to about 5%, and more preferably from about 2% to about 3%.

The disclosure provides for a kit comprising any of the compositions described herein. For example, in some aspects, the topical pharmaceutical composition is packaged in one or more 5 g doses. The packaging of the individual 5 g doses may be by any suitable means, using any suitable container. Suitable containers include, but are not limited to, tubes, tubs, bottles, jars, and vials. The number of individual 5 g doses included in the kit is not particularly limited. In some aspects, the kit contains a two-week supply of individual 5 g doses. In some aspects, the kit contains a one-month supply of individual 5 g doses. In some aspects, the kit further comprises one or more of dosing instructions, an applicator, and a dosing diary.

The disclosure provides a method for treating recurrent aphthous stomatitis, comprising topically administering to a patient in need of treatment a pharmaceutical composition comprising a therapeutically effective amount of PTX. In some aspects, the method further comprises a method of treatment of recurrent aphthous stomatitis that includes the recommendation to restrict smoking and hot beverages such as coffee and tea up to one hour after administering the topical PTX composition.

In some aspects, the composition comprises about 5% (w/w) PTX, about 1.3% (w/w) polyethylene oxide, about 3% (w/w) hypromellose, about 0.8% (7:3 w/w) methylparaben:propylparaben, and distilled water q.s. 100% (w/w).

All aspects described with reference to one aspect apply to all other aspects with equal force, and vice versa.

Aspects of the disclosure are further illustrated by means of the following examples, which are not limiting in any respect. All references are incorporated herein by reference.

### Example 1

This example provides an exemplary formulation described herein.

| Ingredient | % w/w |
|---|---|
| Pentoxifylline | 5.0 |
| Polyethylene Oxide | 1.3 |
| Hypromellose | 3% |
| Methylparaben:Propylparaben (7:3) | 0.8% |
| Q.S. Water | 100% |

### Example 2

This example provides an exemplary formulation described herein.

| Ingredient | % w/w Range |
|---|---|
| Pentoxifylline | 50% |
| Polyethylene Oxide | 0.1-5% |
| Hypromellose | 0.1-10% |
| Methyparaben:Propylparaben | 0.1-5% |
| Q.S. Water | 100% |

### Example 3

### SUMMARY

A randomized, open-label, proof-of-concept clinical study was conducted under an FDA Investigational New Drug Application (IND). 41 patients were randomized to the standard of care (colchicine, CLC-only) or standard of care plus topical PTX (i.e., CLC+PTX) using a pentoxifylline formulation substantially similar to the exemplary formulation in Example 1 (i.e., a muco-adherent gel). Enrolled patients had a least one new oral ulcer within the previous 48 hours, and the largest ulcer was registered as the index ulcer. Subjects in the CLC+PTX group were instructed to apply a 20 ml tube containing 1000 mg topical PTX at least four times/day to all ulcers and to consume the entire tube daily. The CLC was administered orally as a pill and dosed as determined by the subject's physician. No less than every 48 hours, subjects' ulcers were photographed and examined using digital image processing to determine ulcer area, over a period of 14 days, which include daily collection of adverse events, ulcer number, and pain scores.

At enrollment (day zero), the 18 patients randomized to PTX+CLC and the 21 randomized to CLC-only were similar with regard to demographics, disease extent and index ulcer area. Two days after beginning therapy, ulcers began decreasing in area for 15 (83%) of the patients treated with PTX+CLC but only for 4 (19%) of the patients on CLC-only (p<0.001). By day 4, the index ulcer was undetectable for 13 (72%) of the patient treated with PTX+CLC compared to only 6 (29%) of the patients on CLC-only (p<0.01). Curves of mean index ulcer area declined faster and more completely for PTX+CLC compared to CLC only. The daily average number of oral ulcers during the treatment phase was significantly lower for PTX+CLC compared to CLC-only (0.82+/−0.75 vs 1.89+/−1.36, p<0.01). Declines in pain score strongly favored treatment with PTX+CLC.

Surprisingly, compared to standard of care, topical PTX appears to have stronger immediate effects on ulcer healing, faster effects on ulcer clearance, and lessening effects on ulcer number. Findings on pain scores were also supportive. This first controlled trial of topical PTX for the treatment of BD oral ulcers yielded strongly encouraging results.

### Methods

A randomized, open-label, proof-of-concept study of PTX compounded into a muco-adherent gel and topically applied to the oral ulcers of BD patients was conducted. Inclusion criteria required the recent (<2 days) appearance of a new oral ulcer. Patients on immunosuppressant therapies were excluded from the study. Patients were randomized to either standard of care (CLC-only) or standard of care plus treatment with 5% PTX gel (PTX+CLC), orally applied to all existing oral ulcers no less than four times daily using one tube per day (containing 1000 mg PTX). All standard good clinical practice (GCP) procedures for adverse event reporting and monitoring of patient compliance were followed.

Patients were contacted daily for 14 consecutive days and were examined in-person no less than every 48 hours except for weekends. On the first day of entry into the study, the largest ulcer was identified and photographed as the “index ulcer”; its location was recorded and it was subsequently photographed at every in-person visit. Photographs were made with a paper ruler showing millimeter graduations lain in the same plane as the ulcer itself. Graphical processing software was used to compute the ulcer area in square millimeters; this software was calibrated from the paper ruler image and required drawing a line around the image of the ulcer margin. At all daily contacts (both in-person and by telephone) patients reported their pain experience on a scale of 1 to 10.

Treatment groups (PTX+CLC vs. CLC-only) were compared in the growth/shrinkage of their index ulcers. First, the direction of size change (grew or shrank) that index ulcer had over the first two days of treatment was compared. Then, the proportion of patients with undetectable index ulcer (defined as those with an ulcer area of less than 1 mm²) at day 4 was compared. Lastly, the trend in mean ulcer area over 14 days was compared as well as the results' sensitivity to outliers.

The total number of oral ulcers were also counted at each in-person visit, and the daily average number of oral ulcers were calculated during the treatment period.

The average day zero pain score for each treatment group was normalized to 100%. Then for each treatment group averages were plotted for succeeding days as a percentage of the first day's pain score average.

### Results

### Patient Demographics

Of 41 patients enrolled in the study, 20 were randomized to PTX+CLC and 21 were randomized to CLC-only. Two patients from the PTX+CLC group were withdrawn due to a non-serious adverse event (nausea). Table 1 shows that patients in these two groups were similar with regard to age (median of 39.4 vs 39), percent female (55% vs 52%), mean duration since BD diagnosis (6.1 years vs 6.5 years), history of genital ulcers (90% vs 95%), and ocular involvement (14% vs 10%). On day zero, patients had similar mean index ulcer area (6.8+/−7.7 vs 6.3+/−7.4). While patients randomized to the CLC-only group had more ulcers on day zero (3+/−2.1 vs 1.9+/−0.9), this difference was not statistically significant.

TABLE 1
Patient Demographics

| | PTX + CLC | | CLC | |
|---|---|---|---|---|
| | (n = 18) | | (n = 21) | |
| Age -Years | | | | |
| Mean (Median) | 39.5 | (39.4) | 39.7 | (39) |
| Range | 20-54 | | 19-58 | |
| Sex - Number (%) | | | | |
| Female | 11 | (55%) | 11 | (52%) |
| Male | 7 | (45%) | 10 | (48%) |
| Duration Since Behçet's | 6.1 | | 6.5 | |
| Disease Diagnosis-Years | | | | |
| Behçet's Disease | | | | |
| Extent/Patient | | | | |
| History-Number (%) | | | | |
| Patients with Oral Ulcers | 18 | (100%) | 21 | (100%) |
| Patients with Genital Ulcers | 17 | (95%) | 19 | (90%) |
| Patients with Ocular Symptoms | 2 | (10%) | 3 | (14%) |
| Ulcers at Day 0 | | | | |
| Index Ulcer Area-mm² | | | | |
| Mean | 6.3 | (+/−7.4) | 6.8 | (+/−7.7) |
| (Standard Deviation) | | | | |
| Mean Difference | 0.5 | (−4.4 to 5.4) | | |
| (95% Confidence Interval) | | | | |
| Total Oral Ulcer Number | | | | |
| Mean | 1.9 | (+/−0.9) | 3 | (+/−2.1) |
| (Standard Deviation) | | | | |
| Mean Difference | 1.1 | (0 to 2.2) | | |
| (95% Confidence Interval) | | | | |

### Safety

All adverse events were reviewed in real time by the treating physician investigator. Among patients treated with PTX+CLC, reports of nausea and dysgeusia (bad taste in mouth) were highly correlated. Dysgeusia was reported among 11 patients (55%) and nausea reported among 15 patients (75%) treated with PTX+CLC. Corresponding numbers for CLC-only were 0 (0%) and 2 (10%). Vomiting occurred among 2 patients treated with PTX+CLC (10%) but none with CLC-only. No serious adverse events occurred.

Two patients withdrew from the study, both were assigned to topical PTX+CLC and both were withdrawn due to unacceptable dysgeusia and nausea (one with vomiting).

Other recorded adverse events included ill-defined oral paresthesias which were not translatable between Turkish and MedDRA coding systems. Additionally, limited dermal rashes were reported among one (6%) patient treated with PTX+CLC and 6 (29%) treated with CLC-only. Rashes were interpreted by the attending physician to represent “confounding by indication” from the underlying disease (i.e. dermal manifestations of BD).

### Index Ulcer Healing Comparisons

Table 2 shows how ulcers changed size at day 2 and 4. At day 2, index ulcers for 15 of 18 (83%) patients treated with PTX+CLC decreased in size, while 4 of 21 (19%) patients treated with CLC-only decreased in size (p<0.001). At day 4, 13 of 18 (72%) PTX+CLC patients had undetectable ulcers compared to 6 of 21 (29%) for CLC-only patients (p<0.01).

TABLE 2
Index Ulcer Healing Analysis

| | PTX + CLC | | CLC | |
|---|---|---|---|---|
| | (n = 18) | | (n = 21) | |
| Day 2 Growth/Shrinkage-Number (%) | | | | |
| Patients with Index Ulcer Shrinkage | 15 | (83%) | 4 | (19%) |
| Patients with Index Ulcer Growth | 3 | (17%) | 16 | (80%) |
| Day 4 Clearance-Number (%) | | | | |
| Patients with Undetectable Ulcers | 13 | (72%) | 6 | (29%) |
| Patients with Remaining Ulcers | 5 | (28%) | 15 | (71%) |

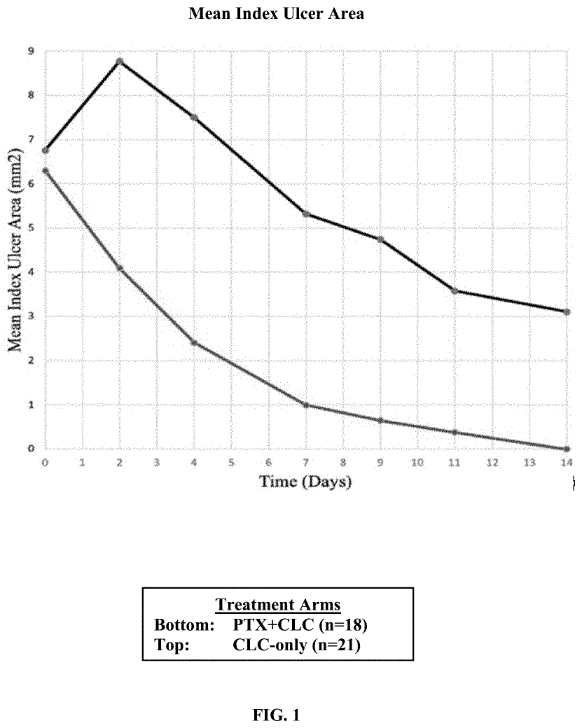

FIG. 1 shows the mean index ulcer area by treatment group. At day zero, index ulcer was similar for both groups, but mean index ulcer area immediately decreased for PTX+CLC, while first growing then slowly decreasing for CLC-only.

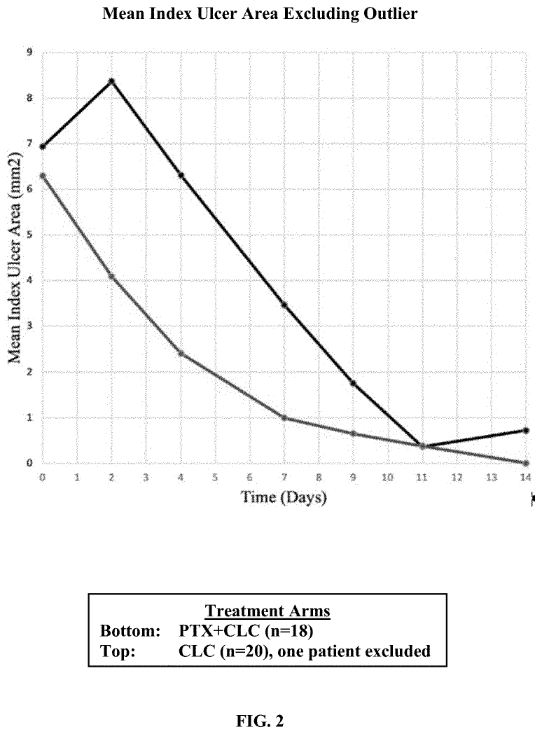

One patient treated with CLC-only experienced dramatic ulcer growth. To test the sensitivity of our analysis to this one data point, the outlier was removed in the graph shown in FIG. 2. However, considerable treatment group differences persisted.

### Analysis of Ulcer Numbers

Another way or measuring ulcer clearance is to analyze the daily average number of ulcers over the two weeks of treatment. Patients treated with PTX+CLC had a significantly lower daily average number of ulcer over the treatment period (0.82+/−0.75 vs 1.89+/−1.36, p<0.05).

### Pain Measurements

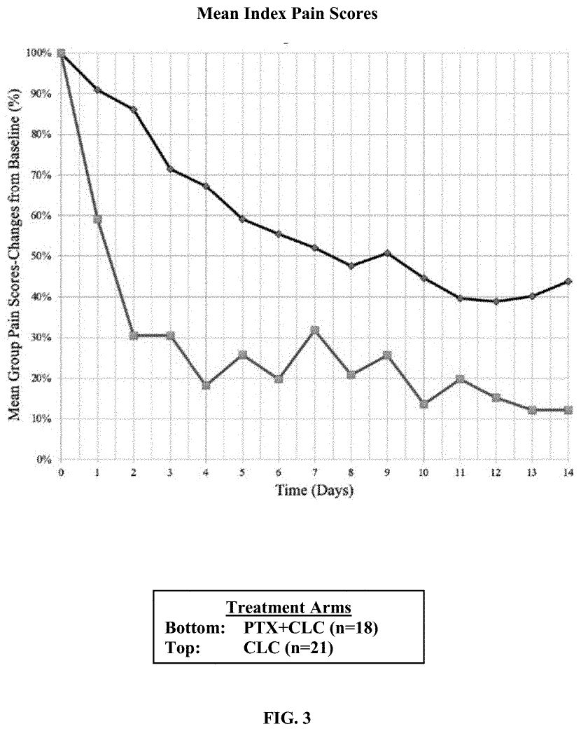

As FIG. 3 demonstrates, normalized pain scores likewise decreased faster and remained lower for PTX+CLC compared to CLC alone. Other analyses (not shown) confirmed similar trends for pain scores specific to eating, drinking, speaking and daily dental care.

### CONCLUSION

The clinical data show that topical PTX therapy accelerates the healing of Behçet's Disease oral ulcers compared to the standard of care (CLC-only). Randomization provided treatment and control groups that were very similar prior to beginning the intervention, yet these two groups had highly dissimilar outcomes.

This study was limited by its small size, lack of control for placebo effect and lack of control for any therapeutic effects of our excipients. While randomization yielded demographically similar groups and similar index ulcer size, by chance ulcer number was not similar at day zero. Placebo effect may well have unduly influenced our pain score findings. The potential for therapeutic effects from excipients (and not for PTX itself) must be addressed in future studies. Nevertheless, index ulcer healing surprisingly was much faster for PTX+CLC, which unlikely resulted from placebo effect.

Topical PTX was generally well tolerated, however dysgeusia was noted for most PTX-treated patients. It is difficult to disentangle the nauseating effects of taste from the gastrointestinal effects previously known to accompany systemic (oral) PTX administration. Regardless, few patients dropped out and many sufficiently valued the therapeutics benefits of topical PTX to request continuation of drug supply subsequent to the trial's conclusion.

Significant differences were demonstrated in immediate treatment effects at day 2 and ulcer clearance at day 4 between PTX+CLC and CLC-only. Remarkable differences in speed of healing were not eliminated by the removal of an outlier.

The 2-week study was designed to measure short-term effects on ulcer healing. No differences in daily average number of ulcers was expected. The same analysis demonstrated that PTX reduced the daily average number of ulcers to less than half found in CLC-only, which was an unexpected finding that suggests that an endpoint for daily average number of ulcers may be directly adapted to definitive trials for topical PTX.

BD is a serious illness with lifelong morbidity that affects speech, eating and sex. These effects are so profound as to generate an entire medical literature about suicidal ideation among persons with BD. The data from this study suggests also potential potent effects on the pain of BD oral ulcers, despite the study not being controlled with a placebo.

In conclusion, this first controlled trial of topical PTX for the treatment of BD oral ulcers yielded surprising and strongly encouraging results.

This written description uses examples to disclose the invention, including the preferred embodiments, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. Aspects from the various embodiments described, as well as other known equivalents for each such aspect, can be mixed and matched by one of ordinary skill in the art to construct additional embodiments and techniques in accordance with principles of this application.

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

Thus, the claims are not intended to be limited to the aspects shown herein, but are to be accorded the full scope consistent with the language of the claims.

Reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference. No claim is to construed as a means plus function unless the element is expressly recited using the phrase “means for.

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C.

All references throughout this application, for example patent documents including issued or granted patents or equivalents; patent application publications; and non-patent literature documents or other source material; are hereby incorporated by reference herein in their entireties, as though individually incorporated by reference.

Moreover, nothing disclosed herein is intended to be dedicated to the public, regardless of whether such disclosure is explicitly recited in the claims.

1. A method of treating Behçet's Disease (BD) comprising topically administering a pharmaceutical composition comprising a therapeutically effective amount of pentoxifylline (PTX) to a human in need of treatment.

2. The method of claim 1, wherein the pharmaceutical composition is muco-adherent.

3. The method of claim 1 or 2, wherein the pharmaceutical composition comprises about 5% (w/w) PTX, about 1.3% (w/w) polyethylene oxide, about 3% (w/w) hypromellose, about 0.8% methylparaben:propylparaben (7:3) and distilled water q.s. 100% (w/w).

4. The method of any one of claims 1-3, wherein the pharmaceutical composition is administered in one or more doses of no more than about 5 g each of the pharmaceutical composition.

5. The method of any one of claims 1-4, wherein no more than about 20 g of the pharmaceutical composition is administered per day.

6. The method of any one of claims 1-5, wherein the PTX treats oral ulcers in the human.

7. The method of any one of claims 1-6, wherein the PTX treats genital ulcers in the human.

8. The method of any one of claims 1-7, wherein the pharmaceutical composition is administered at least twice daily.

9. The method of claim 8, wherein the pharmaceutical composition is administered four times daily.

10. A topical oral or genital mucosal pharmaceutical composition comprising:
about 5 (w/w) % pentoxifylline (PTX),
about 1.3 (w/w) % polyethylene oxide,
about 3 (w/w) % hypromellose, and
about 0.8% (w/w) methylparaben:propyl:propylparaben (7:3)
distilled water q.s. 100%.

11. A kit comprising the pharmaceutical composition of claim 10, wherein the pharmaceutical composition is packaged in one or more 5 g doses.

12. A method of treating recurrent aphthous stomatitis, comprising topically administering a pharmaceutical composition comprising a therapeutically effective amount of PTX to a human in need of treatment.

13. The method of claim 12, wherein the pharmaceutical composition is muco-adherent.

14. The method of claim 12 or 13, wherein the pharmaceutical composition comprises about 5% (w/w) PTX, about 1.3% (w/w) polyethylene oxide, about 3% (w/w) hypromellose, about 0.8% (w/w) methylparaben:propylparaben (7:3), and distilled water q.s. 100% (w/w).

15. The method of any one of claims 12-14, wherein the pharmaceutical composition is administered in one or more doses of no more than about 5 g each of the pharmaceutical composition.

16. The method of any one of claims 12-15, wherein no more than about 20 g of the pharmaceutical composition is administered per day.

17. The method of any one of claims 12-16, wherein the pharmaceutical composition is administered at least twice daily.

18. The method of claim 17, wherein the pharmaceutical composition is administered four times daily.

19. A method of treating recurrent genital ulcers, comprising topically administering a pharmaceutical composition comprising a therapeutically effective amount of PTX to a patient in need of treatment.

20. The method of claim 19, wherein the pharmaceutical composition is muco-adherent.

21. The method of claim 20, wherein the pharmaceutical composition comprises about 5% (w/w) PTX, about 1.3% (w/w) polyethylene oxide, about 3% (w/w) hypromellose, about 0.8% (w/w) methylparaben:propylparaben (7:3), and distilled water q.s. 100% (w/w).

22. The method of any one of claims 19-21, wherein the pharmaceutical composition is administered in one or more doses of no more than about 5 g each of the pharmaceutical composition.

23. The method of any one of claims 19-22, wherein no more than about 20 g of the pharmaceutical composition is administered per day.

24. The method of any one of claims 19-23, wherein the pharmaceutical composition is administered at least twice daily.

25. The method of claim 24, wherein the pharmaceutical composition is administered four times daily.

26. The method of claim 1 or 2, wherein said pharmaceutical composition is administered with in conjunction with other oral medications, such as oral colchicine (CLC).

27. The method of claim 26, wherein the pharmaceutical composition is administered at least twice daily.

28. The method of claim 26, wherein the pharmaceutical composition is administered four times daily.

29. A topical oral or genital mucosal pharmaceutical composition comprising pentoxifylline (PTX) administered in conjunction with other oral medications, such as oral colchicine (CLC).

30. The pharmaceutical composition of claim 29, further comprising polyethylene oxide, hypromellose, and distilled water.

31. A method of treating recurrent genital ulcers, comprising topically administering a pharmaceutical composition comprising a therapeutically effective amount of PTX used in conjunction with other oral medications, such as oral colchicine (CLC) to a patient in need of treatment.

32. The method of claim 30, wherein the pharmaceutical composition is muco-adherent.

33. The method of claim 31, wherein the pharmaceutical composition further comprises polyethylene oxide, hypromellose, and distilled water.

34. The method of any one of claims 30-32, wherein the pharmaceutical composition is administered at least twice daily.

35. The method of claim 33, wherein the pharmaceutical composition is administered four times daily.
